(19) United States
(12) Patent Application Publication
(10) Pub. No.: US 2025/0312500 A1
(43) Pub. Date: Oct. 9, 2025

(54) RADIOPHARMACEUTICALS WITH HIGH STABILITY AND RADIOLABELING EFFICIENCY AND THERANOSTIC KIT COMPRISING SAID RADIOPHARMACEUTICALS

(71) Applicants: ECZACIBASI MONROL NÜKLEER ÜRÜNLER SANAYI VE TICARET ANONIM SIRKETI, Gebze/Kocaeli (TR); EGE ÜNIVERSITESI IDARI VE MALI ISLER DAIRE BSK., Bornova/Izmir (TR)

(72) Inventors: Emre ÖZGENÇ, Bornova/Izmir (TR); Evren ATLIHAN GÜNDOGDU, Bornova/Izmir (TR); Zeynep BURAK, Bornova/Izmir (TR); Serkan OBAN, Gebze/Kocaeli (TR)

(21) Appl. No.: 18/864,762

(22) PCT Filed: May 31, 2022
(86) PCT No.: PCT/TR2022/050504

§ 371 Date: Nov. 11, 2024

(30) Foreign Application Priority Data
May 13, 2022 (TR) 2022/007815

(51) Int. Cl. A61K 51/10

(52) U.S. Cl. CPC A61K 51/1051; A61K 51/1093; A61K 2121/00; A61K 2123/00

(57) ABSTRACT

Disclosed are radiopharmaceuticals with high stability and radiolabeling efficiency obtained by attaching a radionuclide to a pharmaceutical part with high stability that is obtained by attaching a chelating agent to a pharmaceutical specific to the receptors present in the tumor area, the radiopharmaceuticals enabling the imaging and/or the destruction of the cancer cells. Also disclosed is are a method for obtaining the radiopharmaceuticals and to a kit including the radiopharmaceuticals. The radiopharmaceuticals basically include at least one radionuclide, which enables the imaging and/or the destruction of the cancer cells, at least one pharmaceutical part, which includes at least one pharmaceutical agent targeting the receptor that causes the growth of the cancer cells, and at least one chelating agent enabling at least one radionuclide to be attached to the pharmaceutical agent.

### SUBJECT OF THE INVENTION

The subject of the invention relates to the radiopharmaceuticals with high stability and radiolabeling efficiency obtained by attaching a radionuclide to a pharmaceutical part with high stability that is obtained by attaching a chelating agent to a pharmaceutical specific to the receptors present in the tumor area in the cancer patients, said radiopharmaceuticals enabling the imaging of the cancer cells without performing an invasive procedure or enabling the destruction of the cancer cells with high efficiency without damaging the surrounding healthy tissues, to a method for obtaining said radiopharmaceuticals and to a kit comprising said radiopharmaceuticals.

### STATE OF THE ART

Cancer is a disease resulting in the uncontrolled growth of the abnormal cells and the formation of a cellular mass known as tumor by these cells. The cancer cells are usually malignant, leading to the tumor formation also in the other parts of the body by spreading via the lymphatic and vascular system and to the death of the patient when they become uncontrollable. Therefore, it is necessary to diagnose the disease in a rapid and accurate manner and treat it with the appropriate treatment methods after diagnosed. The general treatment methods of the state of the art applied to the patient after the cancer is diagnosed via the invasive methods such as biopsy and the non-invasive methods such as imaging include the surgical therapy, chemotherapy and/or radiation therapy. The surgical procedure is an invasive procedure where the tissues are subjected to physical intervention by making incisions.

The targeted treatment methods using the pharmaceuticals and the radionuclides that target only the cancerous tissue are employed for the purpose of destroying the cancer cells without damaging the surrounding healthy tissues, thus without causing the side effects such as nausea, vomiting, hair loss, loss of appetite, mouth ulcer and reduced blood cell count. The radiopharmaceuticals are obtained by way of attaching a radionuclide, which enables the imaging and/or the destruction of the cancer cells, to a tumor-specific pharmaceutical, i.e., by way of labeling the pharmaceutical with a radionuclide. The radiopharmaceuticals reach the cancer cells via the blood circulation, accumulate in these tissues upon the pharmaceutical adhering to the receptors in the cells and enable the imaging and/or the destruction of the cells owing to the radiation emitted on the molecular level by the radionuclide they carry.

Human epidermal growth factor receptor 2 (HER2) is a protein that is available in all the human cells and that controls the growth and repair of the cells. The elevated HER2 levels that support the growth and the survival of the cancer cells are present in some types of breast, esophageal and gastric cancer. These are known as HER2-positive cancers. The invasive methods such as immunohistochemistry (IHC) and fluorescence in situ hybridization (FISH), which are commonly employed for the purpose of detecting the tumors associated with the HER2 receptor, i.e. for the purpose of diagnosing the disease, bring about the necessity to conduct a biopsy to be able to study the tumor size, in which case the diagnosis and the treatment of the disease become difficult. Moreover, said methods fail to give accurate results and reduce the treatment efficiency in cases where the tumor is a heterogeneous tumor consisting of HER2-positive metastatic tumors and HER2-negative primary tumors. The pharmaceutical Trastuzumab, with the known commercial name Herceptin, which provides targeted destruction, has started to be used in the treatment of the breast cancer patients that are HER2-positive; however, using Trastuzumab alone in the treatment causes the development of primary resistance in the patients.

The radiopharmaceuticals are used in order to accurately detect the HER2-positive cancer cells in the body without performing an invasive procedure on the patient and to destroy these cells without damaging the surrounding healthy cells. The monoclonal antibody Trastuzumab forms the pharmaceutical part of the radiopharmaceutical. Trastuzumab blocks the effects of HER2 and stimulates the immune system to attack and kill the cancer cells. The imaging radionuclides, of which Gallium-68 (Ga-68) may be mentioned as an example, are attached to the pharmaceutical in case it is desired to use the radiopharmaceutical for the purpose of imaging the cancer cells and the destructive radionuclides, of which Lutetium-177 (Lu-177) may be mentioned as an example, are attached to the pharmaceutical in case it is desired to use the radiopharmaceutical for the purpose of destroying the cells without damaging the healthy tissues and without causing the development of the primary resistance in the patients.

With the radiopharmaceutical Ga-68-Trastuzumab injected to the patient via intravascular access, the patient's body may be imaged via PET and the presence of the HER2 receptors in the tumor area may be detected without having to perform an invasive procedure. Owing to the radionuclide Ga-68 having a half-life of 68 minutes, the patient does not need to stay in the hospital after the imaging is performed. Further, since the radiopharmaceutical Ga-68-Trastuzumab will reveal all the HER2 receptors in the patient, it will be possible to accurately detect the presence of the primary and metastatic tumors. The radiopharmaceutical Lu-177-Trastuzumab results in the targeted destruction of a tumor by causing minimum damage to the healthy cells. The radionuclide Lu-177 may transmit its energy to the small-sized tumor cells also. Owing to the gamma rays emitted by the radionuclide Lu-177, it becomes possible to detect, by way of imaging via SPECT, whether the radiopharmaceutical has attached to the tumor foci after being administered to the patient. In this way, the success of treatment is increased in the patients with breast cancer and the side effects and the treatment expenses are reduced due to the use of the pharmaceutical at lower doses.

The radionuclides are attached to the pharmaceuticals via ligands/chelating agents. The pharmaceuticals combine with the ligands to form the chelate complexes and may be preserved in this state until they will be labeled with a radionuclide. Said agents affect the efficiency of the labeling of the pharmaceutical with the radionuclide, i.e. the efficiency of obtaining the radiopharmaceutical and the stability of the radiopharmaceutical. The use of a stable radiopharmaceutical with high radiolabeling efficiency reduces the misdiagnosis rate and/or enhances the efficacy of the treatment. The chelating agents such as DOTA and NOTA are commonly used for labeling the pharmaceutical Trastuzumab with the radionuclides such as Ga-68 or Lu-177. Because the cancer is an aggressive disease, has an incidence increasing day by day and causes the death of an increasing number of people, it is desired that the radiopharmaceuticals used for the diagnosis and treatment have the highest level of stability and efficacy possible. Consequently, the chelating agents, which provide higher radiolabeling efficiency and stability than the radiolabeling efficiency and stability achieved with the conventional chelating agents when preparing the radiopharmaceuticals that enable the imaging and the destruction of the HER2-containing cancer cells, and the radiopharmaceuticals, which are prepared with such chelating agents, are needed.

In order to meet the mentioned need, a radiopharmaceutical comprising a pharmaceutical part, which includes the pharmaceutical agent Trastuzumab and the chelating agent mercapto acetyl triglycine-3 (MAG3), and a radionuclide, preferably Ga-68 or Lu-177, has been developed within the scope of the invention, for use in the highly efficient non-invasive diagnosis of especially the HER2 receptor-containing cancer types and in the destruction of the cancer cells without damaging the healthy tissue and without causing the development of primary resistance in the patient. Owing to the chelating agent it contains, said radiopharmaceutical has higher radiolabeling efficiency and thus higher stability and efficacy compared to the conventional radiopharmaceuticals, wherein the radiopharmaceutical Ga-68-MAG3-Trastuzumab enables the detection of the HER2-positive and HER2-negative cancer cells with high reliability, while the radiopharmaceutical Lu-177-MAG3-Trastuzumab enables the removal of the cancer cells from the target area with high efficiency. The possibility to offer the radiopharmaceuticals in the form of a kit to the users is another unique feature of the invention. The theranostic kit enables the radiopharmaceutical to be easily and safely carried and stored and thus enables the same to have a long shelf life.

### Object of the Invention

An object of the invention is to develop a radiopharmaceutical comprising a chelating agent, which enables at least one radionuclide to be attached to a pharmaceutical agent specific to a particular cancer receptor, has high stability and enables the radiolabeling to take place with high efficiency.

Another object of the invention is to develop a radiopharmaceutical comprising a chelating agent, which enables high radiolabeling efficiency and stability values to be obtained even when used in low quantities and which thus reduces the manufacture and procurement costs of the radiopharmaceutical.

Another object of the invention is to develop a radiopharmaceutical, which, owing to its high stability, enables the detection of the cancer cells with higher reliability compared to the conventional radiopharmaceuticals without performing any invasive procedure on the patient.

Another object of the invention is to develop a radiopharmaceutical, which, owing to its high stability, has higher efficacy compared to the conventional radiopharmaceuticals in the destruction of the cancer cells without primary resistance developing in the patient.

Another object of the invention is to develop a theranostic kit, which enables the radiopharmaceutical to be carried and stored more easily and safely and thus enables the radiopharmaceutical to have a long shelf life.

Another object of the invention is to use the compound mercapto acetyl triglycine-3 (MAG3) for the development of the radiopharmaceuticals enabling the imaging and/or the destruction of the cancer cells originating from the HER2 receptor; i.e., to benefit from the superior properties of the compound MAG3 in a new field.

### DESCRIPTION OF THE TABLES

Table 1. Initial particle size, zeta potential and PDI values of the complexes

Table 2. Particle size, zeta potential and PDI values of the complexes kept at a temperature of 5±3° C. for 3 months as measured at the end of this duration

Table 3. Particle size, zeta potential and PDI values of the complexes kept at a temperature of 25±5° C. and a relative humidity of 60±5% for 3 months as measured at the end of this duration

Table 4. Particle size, zeta potential and PDI values of the complexes kept at a temperature of 40±5° C. and a relative humidity of 75±5% for 3 months as measured at the end of this duration

Table 5. Particle size, zeta potential and PDI values of the complexes kept at a temperature of 5±3° C. for 6 months as measured at the end of this duration

Table 6. Particle size, zeta potential and PDI values of the complexes kept at a temperature of 25±5° C. and a relative humidity of 60±5% for 6 months as measured at the end of this duration

Table 7. Particle size, zeta potential and PDI values of the complexes kept at a temperature of 40±5° C. and a relative humidity of 75±5% for 6 months as measured at the end of this duration

Table 8. Radiolabeling efficiencies of the complexes with the radionuclide Ga-68

Table 9. Radiolabeling efficiencies of the complexes with the radionuclide Lu-177

### DESCRIPTION OF THE INVENTION

The subject of the invention relates to the radiopharmaceuticals with high stability and radiolabeling efficiency obtained by attaching a radionuclide to a pharmaceutical part with high stability that is obtained by attaching a chelating agent to a pharmaceutical specific to the receptors present in the tumor area in the cancer patients, said radiopharmaceuticals enabling the imaging of the cancer cells without performing an invasive procedure or enabling the destruction of the cancer cells with high efficiency without damaging the surrounding healthy tissues, to a method for obtaining said radiopharmaceuticals and to a kit comprising said radiopharmaceuticals.

The radiopharmaceutical according to the invention basically comprises at least one radionuclide, which enables the imaging and/or the destruction of the cancer cells, and at least one pharmaceutical part/complex, which includes at least one pharmaceutical agent targeting the receptor that causes the growth of the cancer cells and at least one chelating agent enabling at least one radionuclide to be attached to said pharmaceutical agent.

In some preferred embodiments of the radiopharmaceutical according to the invention, said pharmaceutical agent is a monoclonal antibody.

In a preferred embodiment of the radiopharmaceutical according to the invention, the receptor known to cause the breast or gastric cancer in the cancer cells is the human epidermal growth factor receptor 2 (HER2) and the pharmaceutical part comprises as the pharmaceutical agent the monoclonal antibody Trastuzumab specific to this receptor.

In a preferred embodiment of the radiopharmaceutical according to the invention, said chelating agent is the compound mercapto acetyl triglycine-3 (MAG3), which enables the radiopharmaceutical to have high radiolabeling efficiency and stability.

The pharmaceutical part included in the radiopharmaceutical according to the invention comprises the pharmaceutical agent and the chelating agent in quantities enabling the ratio of the molarity of the pharmaceutical agent to the molarity of the chelating agent to be a value in the range of 0.01-1.

The pharmaceutical part included in the radiopharmaceutical according to the invention comprises the pharmaceutical agent and the chelating agent in quantities enabling the ratio of the molarity of the pharmaceutical agent to the molarity of the chelating agent to be a value of preferably 0.01 or 0.1 or 1.

In a preferred embodiment of the radiopharmaceutical according to the invention, the pharmaceutical part is in the lyophilized form.

The pharmaceutical part included in the radiopharmaceutical according to the invention is obtained by a method comprising the steps of dissolving a certain quantity of the pharmaceutical agent in at least one solvent, dissolving the chelating agent in at least one solvent in a separate medium, combining the solutions and stirring the same for at least 2 hours preferably at a stirring speed of 100-500 rpm and preferably at room temperature, at the end of this duration, incubating the new solution preferably at a temperature of 2-4° C. for 24-48 hours, after this duration, adding to the solution at least one solvent that enables to remove the excess chelating agent in the solution, subjecting the solution to ultra centrifugal filtration preferably at a rate of 2000-5000 rpm for 15-30 minutes, and separating the solution from the filtrate that contains the excess chelating agent. Said ultra centrifugal filtration process is performed with a 30 kDa filter in 3-9 cycles.

The pharmaceutical part in the form of a solution is lyophilized, i.e., freeze-dried, at a temperature of −45° C. and under a pressure of 0.07 mbar for at least 48 hours. The pharmaceutical part preserved in the lyophilized form has greater stability compared to its stability when preserved in the form of a solution. The lyophilized pharmaceutical part may be readily brought into powder form for the radiolabeling process and may be reconstituted rapidly and effectively. Owing to the removal of the water in the ambience via lyophilization, the growth of pathogenic microorganisms is prevented in the pharmaceutical part, for which it is very important to remain sterile.

In order to obtain the pharmaceutical part, the pharmaceutical agent is dissolved in at least one solvent preferably at a ratio of 1 mg/mL and the chelating agent is dissolved in at least one solvent preferably at a ratio of 1 mg/mL, and preferably 1 mL of a 0.2 M solvent enabling to remove the excess chelating agent from the solution is added to the new solution (pharmaceutical part) that is the mixture of the two solutions.

In a preferred embodiment of the radiopharmaceutical according to the invention, sodium bicarbonate is used as the solvent that enables the pharmaceutical agent to be dissolved.

In a preferred embodiment of the radiopharmaceutical according to the invention, sodium bicarbonate is used as the solvent that enables the chelating agent to be dissolved.

In a preferred embodiment of the radiopharmaceutical according to the invention, the buffer HEPES (4-(2-hydroxyethyl)-1-piperazine ethane sulfonic acid) is added to the new solution (pharmaceutical part) that is the mixture of the two solutions, as the solvent enabling the excess chelating agent to be removed from the medium. The ratio of weight of the pharmaceutical part to the volume of said solvent is preferably in the range of 1:1-2:1 weight:volume.

For the embodiment of the radiopharmaceutical according to the invention, which is preferred to be used in the imaging or the destruction of the HER2-associated cancer cells and in which the pharmaceutical part comprises the pharmaceutical Trastuzumab and the chelating agent MAG3, 3 different lyophilized complexes/pharmaceutical parts, referred to as K1, K2 and K3, respectively, were prepared with a ratio of the molarity of the pharmaceutical agent to the molarity of the chelating agent of 0.01, 0.1 and 1, respectively, in order to test how the efficacy and the stability of the pharmaceutical part are affected by the variations in the quantities of pharmaceutical agent and chelating agent, in the time, in the humidity and in the temperature.

In obtaining the complexes, 1 mg/mL sodium bicarbonate was used to enable the pharmaceutical agent to be dissolved, 1 mg/mL sodium bicarbonate was used to enable the chelating agent to be dissolved and 1 mL 0.2 M HEPES (4-(2-hydroxyethyl)-1-piperazine ethane sulfonic acid) buffer was used to remove the excess chelating agent from the medium. The ratio of the weight of the complexes to the volume of the HEPES buffer is 1.

Each lyophilized complex was kept for 3 and 6 months under 3 different ambient conditions, namely at a temperature of 5±3° C., at a temperature of 25±5° C. and a relative humidity of 60±5%, and at a temperature of 40±5° C. and a relative humidity of 75±5%, and the stability of each complex was measured at the end of these durations. For the stability study, the properties of the complexes such as the physical appearance, particle size, zeta potential and polydispersity index (PDI) were evaluated at t=0, i.e. the initial time point, and at 3 and 6 months.

TABLE 1
Initial particle size, zeta potential and PDI values of the complexes

| | Particle size | | Zeta Potential |
|---|---|---|---|
| | (nm ± SD) | PDI | (mV ± SD) |
| K1 | 560.4 ± 123.9 | 0.463 ± 0.080 | −5.61 ± 2.06 |
| K2 | 817.9 ± 129.9 | 0.337 ± 0.068 | −3.30 ± 1.48 |
| K3 | 1126 ± 124.5 | 0.497 ± 0.179 | −11.7 ± 0.723 |

TABLE 2
Particle size, zeta potential and PDI values of the
complexes kept at a temperature of 5 ± 3°
C. for 3 months as measured at the end of this duration

| | Particle size | | Zeta Potential |
|---|---|---|---|
| | (nm ± SD) | PDI | (mV ± SD) |
| K1 | 586.4 ± 5.52 | 0.47 ± 0.11 | −5.58 ± 1.51 |
| K2 | 896.45 ± 5.64 | 0.47 ± 0.08 | −3.62 ± 0.61 |
| K3 | 1108.82 ± 8.68 | 0.27 ± 0.07 | −9.15 ± 1.37 |

TABLE 3
Particle size, zeta potential and PDI values of the complexes
kept at a temperature of 25 ± 5° C. and a relative
humidity of 60 ± 5% for 3 months as measured at the end of this duration

| | Particle size | | Zeta Potential |
|---|---|---|---|
| | (nm ± SD) | PDI | (mV ± SD) |
| K1 | 577.59 ± 21.33 | 0.36 ± 0.21 | −3.41 ± 1.80 |
| K2 | 790.79 ± 2.4 | 0.38 ± 0.22 | −2.34 ± 2.45 |
| K3 | 1102.86 ± 2.14 | 0.28 ± 0.07 | −11.02 ± 1.56 |

TABLE 4
Particle size, zeta potential and PDI values of the complexes
kept at a temperature of 40 ± 5° C. and a relative
humidity of 75 ± 5% for 3 months as measured at the end of this duration

| | Particle size | | Zeta Potential |
|---|---|---|---|
| | (nm ± SD) | PDI | (mV ± SD) |
| K1 | 581.17 ± 13.67 | 0.25 ± 0.18 | −7.08 ± 2.02 |
| K2 | 798.23 ± 9.80 | 0.37 ± 0.12 | −2.88 ± 5.80 |
| K3 | 1117.78 ± 14.53 | 0.28 ± 0.06 | −14.45 ± 3.16 |

TABLE 5
Particle size, zeta potential and PDI values of the
complexes kept at a temperature of 5 ± 3°
C. for 6 months as measured at the end of this duration

| | Particle size | | Zeta Potential |
|---|---|---|---|
| | (nm ± SD) | PDI | (mV ± SD) |
| K1 | 683.13 ± 5.92 | 0.22 ± 0.15 | −8.04 ± 3.81 |
| K2 | 894.9 ± 3.90 | 0.35 ± 0.22 | −3.78 ± 5.4 |
| K3 | 1194.8 ± 4.77 | 0.37 ± 0.26 | −5.52 ± 2.43 |

TABLE 6
Particle size, zeta potential and PDI values of the complexes
kept at a temperature of 25 ± 5° C. and a relative
humidity of 60 ± 5% for 6 months as measured at the end of this duration

| | Particle size | | Zeta Potential |
|---|---|---|---|
| | (nm ± SD) | PDI | (mV ± SD) |
| K1 | 583.04 ± 5.18 | 0.37 ± 0.23 | −6.48 ± 2.55 |
| K2 | 794.79 ± 5.04 | 0.38 ± 0.20 | −2.23 ± 1.18 |
| K3 | 1103.14 ± 6.55 | 0.33 ± 0.31 | −13.92 ± 3.96 |

TABLE 7
Particle size, zeta potential and PDI values of the complexes
kept at a temperature of 40 ± 5° C. and a relative
humidity of 75 ± 5% for 6 months as measured at the end of this duration

| | Particle size | | Zeta Potential |
|---|---|---|---|
| | (nm ± SD) | PDI | (mV ± SD) |
| K1 | 576.07 ± 5.21 | 0.37 ± 0.04 | −5.88 ± 4.72 |
| K2 | 903.23 ± 15.90 | 0.25 ± 0.17 | −4.00 ± 3.61 |
| K3 | 995.56 ± 4.84 | 0.34 ± 0.29 | −10.31 ± 2.86 |

It was noted as a result of the measurements that the variations in the values of the quantity of the pharmaceutical agent, the quantity of the chelating agent, the temperature and the humidity did not cause a significant change in the particle size, zeta potential and PDI values of the complexes. In addition, the PDI values remained below 0.5 in each of the time frames. This is a value evidencing that the complexes were prepared homogeneously. This in turn enables the complexes and the radiopharmaceuticals comprising said complexes to have high efficacy. It was understood as a result of the measurements that all the complexes comprising the pharmaceutical agent and the chelating agent at different molar ratios had the targeted high stability, and therefore, all the complexes were possible to be radiolabeled with high efficiency and were suitable for use in obtaining the radiopharmaceuticals with high stability.

The radiopharmaceutical according to the invention, in case it is a radiopharmaceutical used for imaging the cancer cells, may comprise at least one radionuclide enabling the cancer cells to be imaged, selected from but not limited to the radionuclides 99 mTc, 168 Re, 186 Re, 153 Sm, 67 Ga, 68 Ga, 69 Ga, 111 In, 59 Fe, 63 Zn, 52 Fe, 45 Ti, 60 Cu, 61 Cu, 67 Cu, 64 Cu, 62 Cu, 198 Au, 199 Au, 195m Pt, 191m Pt, 193m Pt, 117 mSn, 89 Zr, 177 Lu, 16 F, 123 I.

In a preferred embodiment, the radiopharmaceutical according to the invention comprises Ga-68 as the radionuclide that enables the imaging of the cancer cells. Owing to the radionuclide Ga-68 having a half-life of 68 minutes, the patient does not need to stay in the hospital after the imaging is performed. Further, since the radiopharmaceutical Ga-68-Trastuzumab will reveal all the HER2 receptors in the patient, it will be possible to obtain accurate results about the primary and metastatic tumors.

The radiopharmaceutical according to the invention, in case it is a radiopharmaceutical used for destroying the cancer cells, may comprise at least one radionuclide enabling the cancer cells to be destroyed, selected from but not limited to the radionuclides 188 Re, 186 Re, 153 Sm, 166 Ho, b 90 Y, 89 Sr, 111 In, 153 Gd, 225 Ac, 212 Bi, 213 Bi, 211 At, 60 Cu, 61 Cu, 67 Cu, 64 Cu, 62 Cu, 198 Au, 199 Au, 195m Pt, 193m Pt, 197 Pt, 117m Sn, 103 Pd, 103m Rh, 177 Lu, 223 Ra, 224 Ra, 227 Th, 32 P, 161 Tb and 33 P, 125 I, 203 Pb, 201 Ti, 119 Sb, 58m Co, 161 Ho.

In a preferred embodiment of the invention, the radiopharmaceutical comprises Lu-177 as the radionuclide that enables the destruction of the cancer cells. The radiopharmaceutical Lu-177-Trastuzumab results in the targeted destruction of the tumor by causing minimum damage to the healthy cells. The radionuclide Lu-177 may transmit its energy to the small-sized tumor cells also. Owing to the gamma rays emitted by the radionuclide Lu-177, it becomes possible to detect, by way of imaging via SPECT, whether the radiopharmaceutical has attached to the tumor foci after being administered to the patient. In this way, the success of treatment is increased in the cancer patients and the side effects and the treatment expenses are reduced due to the use of the pharmaceutical at lower doses.

The subject of the invention also relates to a method for obtaining the radiopharmaceutical, wherein said method comprises, in addition to the method steps disclosed in the preceding paragraphs for obtaining the pharmaceutical part including the pharmaceutical agent and the chelating agent, the steps of adding to the pharmaceutical part the radionuclide with an activity of 5 mCi in a quantity so that the ratio of the weight of the pharmaceutical part to the volume of the radionuclide will be 1:1-1:3 (weight of the pharmaceutical part:volume of the radionuclide), stirring the solution for at least 1 minute preferably in a vortex type stirrer and, after this duration, keeping the solution at room temperature for at least 1 hour. In a preferred embodiment where the pharmaceutical part is in lyophilized form, the method according to the invention comprises the step of dissolving the pharmaceutical part in at least one solvent prior to the addition of the radionuclide. 1 mL 0.2 M HEPES buffer is preferably used as the solvent that enables the preparation of the solution. The ratio of the weight of the pharmaceutical part to the volume of said buffer is in the range of 1:1-2:1 (weight:volume).

For the embodiments of the radiopharmaceutical according to the invention comprising the pharmaceutical part Trastuzumab-MAG3 and enabling the imaging of the cancer cells, 3 different lyophilized complexes/pharmaceutical parts prepared under the normal conditions with a ratio of the molarity of the pharmaceutical agent to the molarity of the chelating agent of 0.01, 0.1 and 1, respectively, were labeled with the same quantity of the radionuclide Ga-68 with the activity of 5 mCi, in order to test how the radiolabeling efficiency with Ga-68 is affected by the variations in the quantities of the pharmaceutical agent and the chelating agent included in the pharmaceutical part and by the variations in the time. The ratio of the weight of the pharmaceutical part to the volume of the radionuclide is a value in the range of 1:1-1:3 (weight:volume). The pH of the radiopharmaceuticals was measured as 7. The prepared radiopharmaceuticals were named R1, R2 and R3, respectively. The radionuclide Ga-68 has a half-life of 68 minutes. Accordingly, the variation in the radiolabeling efficiencies of these radiopharmaceuticals prepared with Ga-68 was measured for the duration of 60 minutes. The measurement results are provided in Table 8 below.

TABLE 8
Radiolabeling efficiencies of the complexes
with the radionuclide Ga-68

| | Trastuzumab-MAG3-Ga-68 | | |
|---|---|---|---|
| | R1 | R2 | R3 |
| 0 | 94.93 ± 0.65 | 96.02 ± 1.36 | 94.24 ± 0.49 |
| 5 | 93.76 ± 0.75 | 96.1 ± 0.45 | 94.16 ± 0.25 |
| 15 | 94.14 ± 0.71 | 95.14 ± 0.42 | 94.23 ± 0.89 |
| 30 | 93.88 ± 0.25 | 95.51 ± 0.86 | 94.15 ± 0.5 |
| 60 | 94.11 ± 0.78 | 95.44 ± 0.94 | 93.09 ± 1.37 |

For the embodiments of the radiopharmaceutical according to the invention comprising the pharmaceutical part Trastuzumab-MAG3 and enabling the destruction of the cancer cells, 3 different lyophilized complexes/pharmaceutical parts prepared under the normal conditions with the ratio of the molarity of the pharmaceutical agent to the molarity of the chelating agent being 0.01, 0.1 and 1, respectively, were labeled with the same quantity of the radionuclide Lu-177 with the activity of 5 mCi, in order to test how the radiolabeling efficiency with Lu-177 is affected by the variations in the quantities of the pharmaceutical agent and the chelating agent included in the pharmaceutical part and by the variations in the time. The ratio of the weight of the pharmaceutical part to the volume of the radionuclide is a value in the range of 1:1-1:3 (weight:volume). The pH of the radiopharmaceuticals was measured as 7. The prepared radiopharmaceuticals were named R4, R5 and R6, respectively. The radionuclide Lu-177 has a half-life of 7 days. Accordingly, the radiolabeling efficiencies of these radiopharmaceuticals prepared with Lu-177 were measured for the duration of 7 days. The measurement results are provided in Table 9 below.

TABLE 9
Radiolabeling efficiencies of the complexes
with the radionuclide Lu-177

| | Trastuzumab-MAG3-Lu-177 | | |
|---|---|---|---|
| | R4 | R5 | R6 |
| 0 | 95.18 ± 0.14 | 96.92 ± 0.09 | 95.40 ± 0.13 |
| 1 | 95.31 ± 0.03 | 97.08 ± 0.11 | 95.59 ± 0.23 |
| 3 | 94.63 ± 0.31 | 97.23 ± 0.25 | 94.26 ± 0.86 |
| 5 | 93.71 ± 0.16 | 97.38 ± 0.25 | 93.60 ± 0.29 |
| 7 | 93.69 ± 0.79 | 97.04 ± 0.40 | 93.23 ± 0.42 |

Looking at these measurement results, it can be seen that the radiolabeling efficiencies of the complexes, which comprise the chelating agent MAG3 at different ratios, with the radionuclides Lu-177 and Ga-68 were above 95% at the initial time point and were above 93% even at the end of the half-lives of the radionuclides. Therefore, it is understood that MAG3 has high compatibility with the pharmaceutical agent and the radionuclides, the radiolabeling can be performed with high efficiency at any time during the half-life of the radionuclide and the novel pharmaceutical part and the radiopharmaceuticals prepared with this chelating agent also have high stability. Accordingly, unlike the conventional radiopharmaceuticals, the radiopharmaceuticals such as Trastuzumab-MAG3-Ga-68 and Trastuzumab-MAG3-Lu-177, which comprise the compound MAG3, enable the diagnosis of the cancer cells with high reliability and the destruction of the cancer cells with high efficiency. The ability of MAG3, even at very low quantity, to increase the radiolabeling efficiency to the range of 93-97% and to maintain the same within this range throughout the half-life of the radionuclide provides a reduction in the manufacture and procurement costs of the radiopharmaceuticals.

The subject of the invention further relates to a theranostic kit, which comprises at least one radiopharmaceutical according to the invention used for the imaging of the cancer cells and at least one other radiopharmaceutical according to the invention used for the destruction of the cancer cells. A theranostic kit, which comprises at least one radiopharmaceutical according to the invention used both for the imaging of the cancer cells and the destruction of the cancer cells, is also included within the scope of the invention.

The radiopharmaceutical included in the kit according to the invention and used for the imaging of the cancer cells comprises at least one radionuclide, which enables the imaging of the cancer cells, and at least one pharmaceutical part, which is to be radiolabeled with said radionuclide and which is obtained by attaching the chelating agent to the (targeted) pharmaceutical agent specific to the carcinogenic receptors.

The radiopharmaceutical included in the kit according to the invention and used for the destruction of the cancer cells comprises at least one radionuclide, which enables the destruction of the cancer cells, and at least one pharmaceutical part, which is to be radiolabeled with said radionuclide and which is obtained by attaching the chelating agent to the (targeted) pharmaceutical agent specific to the carcinogenic receptors.

The radiopharmaceutical included in the kit according to the invention and used both for the imaging and the destruction of the cancer cells comprises at least one radionuclide, which enables the imaging and the destruction of the cancer cells, and at least one pharmaceutical part, which is to be radiolabeled with said radionuclide and which is obtained by attaching the chelating agent to the (targeted) pharmaceutical agent specific to the carcinogenic receptors.

In a preferred embodiment of the kit according to the invention, the radiopharmaceutical used for the imaging of the cancer cells comprises the radionuclide Ga-68, which enables the imaging of the cancer cells, and at least one pharmaceutical part, which is to be radiolabeled with said radionuclide, includes the pharmaceutical agent Trastuzumab and the chelating agent MAG3 and is preferably in lyophilized form.

In a preferred embodiment of the kit according to the invention, the radiopharmaceutical used for the destruction of the cancer cells comprises the radionuclide Lu-177, which enables the destruction of the cancer cells, and at least one pharmaceutical part, which includes the pharmaceutical agent Trastuzumab and the chelating agent MAG3 and is preferably in lyophilized form.

In a preferred embodiment of the kit according to the invention, the radiopharmaceutical used both for the imaging and the destruction of the cancer cells comprises the radionuclide Lu-177, which enables the imaging and the destruction of the cancer cells, and at least one pharmaceutical part, which includes the pharmaceutical agent Trastuzumab and the chelating agent MAG3 and is preferably in lyophilized form.

The kit comprising the disclosed radiopharmaceuticals enables the disease to be non-invasively diagnosed with high efficiency especially for the breast cancer patients that are HER2-positive and enables the cancer cells to be destroyed without damaging the healthy tissue and without causing the development of primary resistance in the patient. Of the radiopharmaceuticals having high stability and efficacy owing to the chelating agent mercapto acetyl triglycine-3 (MAG3) they contain, which chelating agent enables the radiolabeling to be performed with high efficiency and has high stability, the radiopharmaceutical Trastuzumab-MAG3-Ga-68 enables the HER2-positive and HER2-negative cancer cells to be detected with high reliability and the radiopharmaceutical Trastuzumab-MAG3-Lu-177 enables the HER2-positive and HER2-negative cancer cells to be removed from the target area with high efficiency. The possibility to offer the radiopharmaceutical in the form of a kit is another unique feature of the invention. The theranostic kit enables the radiopharmaceutical to be easily and safely carried and stored and thus enables the same to have a long shelf life.

1. A radiopharmaceutical used for the imaging and/or the destruction of the cancer cells wherein the radiopharmaceutical comprises
at least one radionuclide, which enables the imaging and/or the destruction of the cancer cells, wherein the radionuclide which enables the imaging of the cancer cells is Ga-68 and/or the radionuclide which enables the destruction of the cancer cells is Lu-177;
at least one pharmaceutical part, which includes at least one pharmaceutical agent targeting the receptor that causes the growth of the cancer cells and at least one chelating agent enabling at least one radionuclide to be attached to said pharmaceutical agent, wherein the ratio of the weight of the pharmaceutical part to the volume of the radionuclide is 1:1-1:3 (weight:volume).

2. (canceled)

3. (canceled)

4. (canceled)

5. (canceled)

6. (canceled)

7. A radiopharmaceutical according to claim 1, wherein the pharmaceutical agent is a monoclonal antibody.

8. A radiopharmaceutical according to claim 1, wherein the pharmaceutical agent is a pharmaceutical agent targeting the human epidermal growth factor receptor 2 (HER2).

9. A radiopharmaceutical according to claim 1, wherein the pharmaceutical agent is Trastuzumab.

10. A radiopharmaceutical according to claim 1, wherein the chelating agent is mercapto acetyl triglycine-3 (MAG3).

11. (canceled)

12. (canceled)

13. (canceled)

14. A radiopharmaceutical according to claim 1, wherein the ratio of the molarity of the pharmaceutical agent to the molarity of the chelating agent is in the range of 0.01-1.

15. (canceled)

16. (canceled)

17. (canceled)

18. A radiopharmaceutical according to claim 1, wherein the pharmaceutical part is in the lyophilized form.

19. A theranostic kit comprising:
at least one radiopharmaceutical used for imaging the cancer cells comprising at least one radionuclide which enables the imaging of the cancer cells, is the radionuclide Ga-68, and at least one other radiopharmaceutical used for destroying the cancer cells comprising at least one radionuclide which enables the destruction of the cancer cells, is the radionuclide Lu-177, or
at least one radiopharmaceutical used both for imaging and destroying the cancer cells comprising at least one radionuclide which enables both the imaging and the destruction of the cancer cells is the radionuclide Lu-177.

20. A kit according to claim 19, wherein the radiopharmaceutical used for imaging the cancer cells and/or used for destroying the cancer cells comprises at least one radionuclide, and at least one pharmaceutical part, which is obtained by attaching at least one chelating agent to at least one pharmaceutical agent that targets the receptor causing the growth of the cancer cells.

21. A kit according to claim 20, wherein the pharmaceutical part comprises the pharmaceutical agent Trastuzumab targeting the HER2 receptor and the chelating agent MAG3.

22. (canceled)

23. (canceled)

24. (canceled)

25. (canceled)

26. A kit according to claim 9, wherein the pharmaceutical part is in the lyophilized form.

27. A method for obtaining a radiopharmaceutical used for the imaging and/or the destruction of the cancer cells, the method comprising the steps of:
dissolving a pharmaceutical agent in at least one solvent,
dissolving a chelating agent in at least one solvent in a separate medium,
combining the solutions and stirring the combined solutions for at least 2 hours preferably at a stirring speed of 100-500 rpm and preferably at room temperature,
incubating the solution obtained at the end of this duration preferably at a temperature of 2-4° C. preferably for 24-48 hours (incubation),
at the end of this duration, adding to the solution at least one solvent that enables to remove the excess chelating agent in the solution,
subjecting the solution to ultra centrifugal filtration preferably at a rate of 2000-5000 rpm preferably for 15-30 minutes,
obtaining the pharmaceutical part by removing the filtrate that contains the excess chelating agent,
adding at least one radionuclide, which enables the imaging and/or the destruction of the cancer cells, to the pharmaceutical part,
stirring the mixture containing the pharmaceutical part and the radionuclide for at least 1 minute.

28. (canceled)

29. A method according to claim 27, wherein the ratio of the weight of the pharmaceutical agent to the volume of the solvent is 1 mg/mL.

30. (canceled)

31. (canceled)

32. (canceled)

33. (canceled)

34. (canceled)

35. (canceled)

36. (canceled)

37. (canceled)

38. (canceled)

39. A method according to claim 27, wherein the pharmaceutical part is dissolved in a quantity in the solvent such that the ratio of weight of the pharmaceutical part to the volume of the solvent will be in the range of 1:1-2:1 (weight:volume).

40. (canceled)

41. (canceled)

42. A method according to claim 27, wherein the radionuclide is added in a quantity to the pharmaceutical part such that the ratio of the weight of the pharmaceutical part to the volume of the radionuclide will be in the range of 1:1-1:3 (weight of the pharmaceutical part:volume of the radionuclide).

43. (canceled)

44. (canceled)
